(19) United States
(12) Patent Application Publication
(10) Pub. No.: US 2024/0043984 A9
(43) Pub. Date: Feb. 8, 2024

(54) METHODS FOR FORMING MICROSCALE AND/OR NANOSCALE STRUCTURES ON SURFACES AND DEVICES INCLUDING BIOMEDICAL DEVICES HAVING SURFACES WITH SUCH STRUCTURES

(71) Applicant: Purdue Research Foundation, West Lafayette, IN (US)

(72) Inventors: Kenneth Henry Sandhage, Lafayette, IN (US); Ben M. deGlee, Portland, OR (US)

(21) Appl. No.: 18/000,441

(22) PCT Filed: Jun. 3, 2021
(86) PCT No.: PCT/US2021/035723

§ 371 Date: Dec. 1, 2022

Related U.S. Application Data
(60) Provisional application No. 63/034,671, filed on Jun. 4, 2020.

(51) Int. Cl. C23C 8/12; A61L 27/30

(52) U.S. Cl. CPC C23C 8/12; A61L 27/30

(57) ABSTRACT

Methods for forming micro- and/or nano-structures on the surfaces of a device and devices made thereby. The methods include exposing the surfaces of the device having an initial microstructure to an oxidizing environment at a first elevated temperature so as to form a first oxide scale on the device surfaces, exposing the first oxide scale to a reducing agent at a second elevated temperature so as to convert or partially convert the first oxide scale into a composite scale that includes a second oxide and a first metal, and exposing the composite scale to a dissolution agent that selectively dissolves part or all of the second oxide so as to yield a porous surface layer that includes the first metal.

### BACKGROUND OF THE INVENTION

The present invention generally relates to methods of forming microscale and/or nanoscale structures (micro- and/or nano-structures) on surfaces, and devices having such microscale and/or nanoscale structures on their surfaces. The invention particularly relates to titanium-bearing devices, such as biomedical implants (including dental, maxillofacial, and orthopaedic implants), having surfaces with surface roughnesses and surface porosities tailored by microscale and/or nanoscale structures. Such surfaces include external surfaces of dense (nonporous) titanium-bearing devices, and external and internal surfaces of porous titanium-bearing devices.

Titanium and titanium alloys possess an attractive combination of properties, including biocompatibility, a high strength-to-weight ratio, and corrosion resistance, which make these materials attractive for biomedical applications (e.g., for dental, maxillofacial, and orthopaedic implants). Prior work has shown that titanium-based materials with nanostructures and microstructures on surfaces thereof can exhibit enhanced interactions with bone-forming cells. A variety of methods have been used to develop such nano- and micro-structured surfaces on titanium-based biomedical implants. However, these methods have various limitations including, but not limited to, difficulty controlling the concentration and/or uniformity of the nano- and micro-structures on the surfaces of the titanium-based materials and devices, and reliance on line-of-sight processes (such as grit blasting) for generating nano- and micro-structures on the surfaces of the titanium-based materials and devices.

In view of the above, it can be appreciated that it would be desirable if an improved, non-line-of-sight method was available for producing nano- and/or micro-structures on titanium-based materials and devices, including the ability to control the concentration and/or uniformity of the nano- and micro-structures on surfaces of titanium-based materials and devices, and including the ability to create nano-structures and micro-structures on external surfaces and on internal surfaces of titanium-based materials and devices that cannot be readily accessed by line-of-sight processes.

### BRIEF DESCRIPTION OF THE INVENTION

The present invention provides methods suitable for forming micro- and/or nano-structures on surfaces and devices produced therewith.

According to one aspect of the invention, a non-line-of-sight method is provided for forming micro- and/or nano-structures on a surface of a device. The method includes exposing a surface of the device having an initial microstructure to an oxidizing environment at a first elevated temperature so as to form a first oxide scale on the surface of the device, exposing the first oxide scale to a reducing agent at a second elevated temperature so as to convert or partially convert the first oxide scale into a composite scale that includes a second oxide and a first metal, and exposing the composite scale to a dissolution agent that selectively dissolves part or all of the second oxide so as to yield a porous surface layer that includes the first metal.

According to another aspect of the invention, a device is provided that is produced by a non-line-of-sight method that includes exposing a surface of an initial device having an initial microstructure to an oxidizing environment at a first elevated temperature so as to form a first oxide scale, exposing the first oxide scale to a reducing agent at a second elevated temperature so as to convert or partially convert the first oxide scale into a composite scale that includes a second oxide and a first metal, and exposing the composite scale to a dissolution agent that selectively dissolves part or all of the second oxide so as to yield a porous surface layer that includes the first metal.

Technical effects of the method and device described above preferably include the capability of producing materials and devices having surfaces that include high, uniform concentrations of micro- and/or nano-structures and the capability of producing materials and devices with surfaces, including external and internal surfaces (such as external surfaces of nonporous (dense) materials and devices, and internal surfaces formed by the porosity of porous materials and devices) with high, uniform concentrations of micro- and/or nano-structures by a non-line-of-sight process.

Other aspects and advantages of this invention will be appreciated from the following detailed description.

### BRIEF DESCRIPTION OF THE DRAWINGS

FIG. 1 includes an X-ray diffraction (XRD) analysis of products generated by a reaction of rutile TiO₂ (titania) powder with Mg(g) at 700° C. for 12 hours. Diffraction peaks for MgO (periclase) and α-Ti are labeled with light (grey) and dark (black) lines, respectively.

FIGS. 2A through 2J provide analyses of a dense (nonporous) titanium specimen after an oxidation treatment in ambient air for 6 h at 600° C. FIG. 2A shows a top-down secondary electron (SE) image of the surface of this specimen. FIGS. 2B and 2C show elemental maps for titanium and oxygen, respectively, associated with FIG. 2A. FIG. 2D shows a top-down energy-dispersive X-ray (EDX) analysis of the surface of this specimen. FIG. 2E shows a high-magnification top-down SE image of the surface of this specimen. FIG. 2F shows a scanning transmission electron microscope (STEM) image of an ion-milled cross-section of this specimen. FIGS. 2G and 2H show elemental maps for titanium and oxygen, respectively, associated with FIG. 2F. FIG. 2I shows a selected area electron diffraction (SAED) patterns of an ion-milled cross-section of this specimen. FIG. 2J shows a dark field transmission electron microscope (TEM) image of this specimen revealing rutile crystals.

FIGS. 3A through 3F provide analyses of a dense (nonporous) titanium specimen after an oxidation treatment in ambient air for 6 h at 600° C. and a magnesiothermic reduction treatment for 1 h at 700° C. FIG. 3A shows a top-down SE image of the surface of this specimen. FIGS. 3B, 3C, and 3D show elemental maps for titanium, oxygen, and magnesium, respectively, associated with FIG. 3A. FIG. 3E shows a top-down EDX analysis of the surface of this specimen. FIG. 3F shows a high-magnification top-down SE image of the surface of this specimen.

FIGS. 4A through 4L provide analyses of a dense (nonporous) titanium specimen after an oxidation treatment in ambient air for 6 h at 600° C., a magnesiothermic reduction treatment for 1 h at 700° C., and then selective MgO dissolution in 3 M acetic acid. FIG. 4A shows a top-down SE image of the surface of this specimen. FIGS. 4B, 4C, and 4D show elemental maps for titanium, oxygen, and magnesium, respectively, associated with FIG. 4A. FIG. 4E shows a top-down EDX analysis of the surface of this specimen. FIG. 4F shows a high-magnification top-down SE image of the surface of this specimen. FIG. 4G shows a scanning transmission electron microscope (STEM) image of an ion-milled cross-section of this specimen. FIGS. 4H, 4I, and 4J show elemental maps for titanium, oxygen, and magnesium, respectively, associated with FIG. 4G. FIG. 4K shows a SAED pattern of an ion-milled cross-section of this specimen. FIG. 4L shows a dark field transmission electron microscope (TEM) image of this specimen revealing titanium crystals.

FIG. 5 includes a grazing incidence X-ray diffraction (XRD) analysis of products generated by the reaction of a TiO₂ film with Mg(g) at 700° C. for 24 hours. Diffraction peaks for MgO (periclase) and α-Ti are labeled with light (grey) and dark (black) lines, respectively.

FIGS. 6A and 6B show top-down secondary electron images of a TiO₂ layer formed on a partially oxidized Ti disk (after exposure to flowing air for 1.5 hours at 740° C.; FIG. 6A) and a MgO/Ti layer formed on the same disk surface after complete reaction of the TiO₂ surface layer with flowing Mg(g) for 24 hours at 700° C. (FIG. 6B).

FIGS. 7A, 7B, and 7C show higher magnification top-down secondary electron images of a TiO₂ layer formed on a partially-oxidized Ti disk (after exposure to flowing air for 1.5 hours at 740° C.; FIG. 7A), a MgO/Ti layer formed on the same disk surface after complete reaction of the TiO₂ surface layer with flowing Mg(g) for 24 hours at 700° C. (FIG. 7B), and a porous metallic titanium layer generated on the same disk surface after selective dissolution of the MgO phase in 1M HCl (FIG. 7C).

FIGS. 8A and 8B show higher magnification top-down secondary electron images of a MgO/Ti layer formed on the disk surface after complete reaction of the TiO₂ surface layer with flowing Mg(g) for 24 hours at 700° C. Finer nanoparticles (tens of nm in size, as indicated by the arrow in FIG. 8B) were detected on the surfaces of larger particles (hundreds of nm in size).

FIGS. 9A and 9B show energy dispersive X-ray analyses of a MgO/Ti layer formed on the disk surface after complete reaction of the TiO₂ surface layer with flowing Mg(g) for 24 hours at 700° C. (FIG. 9A), and a porous metallic titanium layer generated by selective dissolution of the MgO phase in 1M HCl for three hours (FIG. 9B).

FIGS. 10A and 10B include top-down secondary electron images of a MgO/Ti layer formed by the complete reaction of a TiO₂ surface layer with flowing Mg(g) for 24 hours at 700° C., and a porous metallic titanium layer formed on the same disk surface (at the same location) after selective dissolution of the MgO phase in 1M HCl, respectively. The selective MgO dissolution generated fine pores detected on the surface in FIG. 10B while retaining themicroscale features and roughness of the MgO/Ti layer of FIG. 10A.

FIGS. 11A through 11N provide analyses of a dense (nonporous) titanium specimen after an oxidation treatment in ambient air for 6 h at 600° C. and a calciothermic reduction treatment for 1 h at 700° C. FIG. 11A shows a top-down SE image of the surface of this specimen. FIGS. 11B, 11C, and 11D show elemental maps for titanium, oxygen, and calcium, respectively, associated with FIG. 11A. FIG. 11E shows a top-down EDX analysis of the surface of this specimen. FIG. 11F shows a high-magnification top-down SE image of the surface of this specimen. FIG. 11G shows a scanning transmission electron microscope (STEM) image of an ion-milled cross-section of this specimen. FIGS. 11H, 11I, and 11J show elemental maps for titanium, oxygen, and calcium, respectively, associated with FIG. 11G. FIG. 11K shows a combined elemental map for titanium oxygen, and calcium associated with FIG. 11G. FIGS. 11L and 11M show SAED patterns obtained from the CaO-rich product layer and a Ti-rich product layer, respectively, in an ion-milled cross-section of this specimen. FIG. 11N shows a dark field TEM image of an ion-milled cross-section of this specimen revealing nanocrystalline CaO and Ti reaction products.

FIGS. 12A through 12H provide analyses of a titanium specimen after an oxidation treatment in ambient air for 6 h at 600° C., a calciothermic reduction treatment for 1 h at 700° C., and then selective CaO dissolution in 3 M acetic acid. FIG. 12A shows a top-down SE image. FIG. 12B shows an elemental map for titanium associated with FIG. 12A. FIG. 12C shows a top-down EDX analysis. FIG. 12D shows a high-magnification top-down SE image. FIG. 12E shows a scanning transmission electron microscope (STEM) image of an ion-milled cross-section. FIG. 12F shows an elemental map for titanium associated with FIG. 12E. FIG. 12G shows a SAED pattern of an ion-milled cross-section. FIG. 12H shows a dark field transmission electron microscope (TEM) image revealing titanium crystals.

FIGS. 13A through 13E provide analyses of a TiAl6V4 specimen after an oxidation treatment in ambient air for 6 h at 600° C. and a calciothermic reduction treatment for 1 h at 700° C. FIG. 13A includes a STEM image of an ion-milled cross-section. FIGS. 13B, 13C, and 13D include elemental maps for titanium, oxygen, and calcium, respectively, associated with the image in FIG. 13A. FIG. 13E shows a SAED pattern of an ion-milled cross-section.

FIGS. 14A through 14E provide analyses of a TiAl6V4 specimen after an oxidation treatment in ambient air for 6 h at 600° C., a calciothermic reduction treatment for 1 h at 700° C., and selective CaO dissolution in 3 M acetic acid. FIG. 14A includes a STEM image of an ion-milled cross-section. FIGS. 14B, 14C, and 14D include elemental maps for titanium, aluminum, and vanadium, respectively, associated with the image in FIG. 14A. FIG. 14E shows a SAED pattern of an ion-milled cross-section.

FIGS. 15A through 15F include secondary electron (SE) images of the surfaces of laser-sintered TiAl6V4 alloy (90 weight % Ti, 6 weight % Al, 4 weight % V) specimens before and after the surface modification process involving the use of a calciothermic reaction process. FIGS. 15A and 15D show the TiAl6V4 specimens in an as-sintered state. FIGS. 15B and 15E show the specimens of FIGS. 15A and 15D, respectively, after (i) 6 hours oxidation in air at 600° C., (ii) 1 hour calciothermic reaction at 700° C., and then (iii) CaO dissolution and ultrasonic cleaning. FIG. 15C shows the specimen of FIG. 15B after laser sintering and polishing to 0.05 μm. FIG. 15F shows the specimen of FIG. 15C after the same reaction treatment as for the specimen in FIG. 15E. Nanoscale roughness was generated, as seen by comparing FIGS. 15D and 15E, and by comparing FIGS. 15C and 15F, while preserving the starting microscale roughness as seen by comparing FIGS. 15A and 15B.

FIG. 16 provides a schematic illustration of the chemical and structural evolution of a Ti alloy surface at various stages of the gas/solid reaction-based (oxidation, calciothermic reaction, dissolution) process.

FIGS. 17A and 17B include graphs representing responses of human female bone marrow stromal cells (MSCs) cultured on TiAl6V4 surfaces with or without a calciothermic reaction (CaR) process (CaR process=oxidation in air at 600° C. for 6 hours, calciothermicreaction at 700° C. for 1 hour, and selective CaO dissolution in 3 M acetic acid for 40 minutes at 25° C.). Responses of MSCs cultured on polished TiAl6V4 specimens without this treatment (“Polished”), polished TiAl6V4 specimens with this treatment (“P-CaR”), grit-blasted and acid-etched TiAl6V4 specimens without this treatment (“Rough”), and grit-blasted and acid-etched TiAl6V4 specimens with this treatment (“R-CaR”) were evaluated. The responses of MSCs cultured on tissue culture polystyrene substrates (TCPS) are also shown. FIG. 17A shows a graph of the osteocalcin analyses from MSCs on the different surfaces. FIG. 17B shows a graph of the osteopontin analyses from MSCs on the different surfaces. Data are shown as the mean±standard error mean, for an N=6 per group, and represent 3 experimental repeats. Statistically significant differences for different surfaces are indicated with different letters.

### DETAILED DESCRIPTION OF THE INVENTION

Disclosed herein are methods suitable for producing devices comprising titanium-based materials that have microscale and/or nanoscale structures (micro- and/or nano-structures) formed on surfaces thereof. As used herein, nanoscale structures, nanoscopic scale structures, or nano-structures are defined as structures comprising geometric features having at least one dimension of about 1 to 300 nanometers, and microscale structures, microscopic scale structures, or micro-structures are defined as structures with geometric features having at least one dimension of about 0.1 to 100 micrometers. Surfaces including the nano-structures and/or micro-structures may be referred to as nano- and micro-structured surfaces (or coatings), respectively. Also disclosed are devices having surfaces with micro- and/or nano-structures formed by these methods including but not limited to titanium-bearing devices for biomedical applications. Such devices produced for biomedical applications may comprise titanium-bearing surfaces or titanium-bearing composite surfaces which can aid in bone formation processes. Also disclosed are devices that are entirely porous or have one or more porous regions so as to have internal surfaces on which micro- and/or nano-structures may be formed by these methods including, but not limited to, titanium-bearing devices with internal surfaces for biomedical applications including, but not limited to, biomedical implant devices for dental, maxillofacial, or orthopaedic applications. Such titanium-bearing devices with internal surfaces include, but are not limited to, porous titanium-bearing dental, maxillofacial, or orthopaedic implants, whose internal porosities define internal surfaces within the devices. Such porous titanium-bearing dental or orthopaedic implants include titanium-bearing dental, maxillofacial, or orthopaedic implants produced by three-dimensional (3-D) printing. Such dental implants include, but are not limited to, implants for teeth. Such maxillofacial implants include, but are not limited to, jawbone implants. Such orthopaedic implants include, but are not limited to, knee, hip, and spinal implants.

Certain previous methods have had difficulty controlling the concentration and/or uniformity of micro- and/or nano-structures formed on surfaces of titanium-based materials. In contrast, the methods disclosed herein utilize well-controlled, non-line-of-sight, displacement reactions including, but not limited to, gas/solid displacement reactions to provide high and uniform concentrations of fine-scale pores and protuberances on surfaces of titanium-based materials. Since the methods are not dependent on line-of-sight processes, the surfaces may include both external and internal surfaces of the bodies. Such internal surfaces include, but are not limited to, internal surfaces within porous regions of titanium-bearing devices produced by three-dimensional (3-D) printing. The sizes of such pores and protuberances can be tailored by the conditions used in the displacement reaction to produce micro- and/or nano-structures.

In certain embodiments, the methods include providing an initial body containing a titanium-bearing compound on at least a surface thereof. A low-temperature reaction is performed on the body that exposes the body to an active reducing agent to allow for reduction of the titanium-bearing compound to yield a titanium-bearing product, preferably elemental titanium and one or more secondary products. A dissolution process may then be performed for complete or partial selective removal of any secondary products of this reaction to yield a layer comprised of porous titanium that retains the shape of the initial body including, for example, macro/microscale features of the initial body. The resulting titanium-bearing products having porous surfaces thereon are especially attractive for biomedical applications including, but not limited to, dental implants, maxillofacial implants, and orthopaedic implants. Such dental implants include, but are not limited to, implants for teeth. Such maxillofacial implants include, but are not limited to, jawbone implants. Such orthopaedic implants include, but are not limited to, knee, hip, and spinal implants.

Chemical reduction approaches (e.g., the magnesiothermic reduction of titania (titanium dioxide, TiO₂)) have been previously reported as means of converting titanium-bearing compounds (such as titania) into metal (elemental) titanium (Ti). However, unlike these previous approaches, the methods herein use a low-temperature chemical reduction process to generate titanium-bearing surfaces with fine-scale pores and protuberances on titanium-bearing substrates, which are believed to be especially beneficial for use in biomedical devices and applications (such as for titanium-based dental, maxillofacial, or orthopaedic implants).

Suitable titanium-bearing compounds include, but are not limited to, oxides. Suitable reducing agents include, but are not limited to, alkaline earth and alkali elements (such as magnesium, calcium, strontium, barium, lithium, sodium, potassium, and/or rubidium). The reducing agent maybe contained within a solid phase or within a fluid phase (wherein the fluid may be a gas, liquid, or plasma). Reduction herein refers to the chemical conversion of the titanium within a titanium-bearing compound (such as a titanium oxide) into elemental titanium.

In certain embodiments, the methods may be used to generate nano- and micro-structured oxide/titanium coatings or nano- and micro-structured porous titanium coatings on titanium or titanium alloy bodies, including bodies with complex shapes such as biomedical implants. To do so, a titanium or titanium alloy body may be first coated with a titanium-bearing compound. Such a coating may be produced via reaction of the body surfaces with a fluid or solid phase. One example of such a reaction-formed compound includes a titanium compound-bearing coating formed by oxidation of the body surfaces with oxygen gas (such as the oxygen present in ambient air) to yield a titanium oxide-bearing (titania-bearing) coating. A titania coating may also be generated on a titanium-bearing body by hydrothermal, or electrochemical oxidation. Alternately, the titanium compound-bearing coating may be applied to the body via the deposition of a titanium-bearing compound by a chemical deposition process (e.g., via surface sol-gel deposition, chemical vapor deposition, atomic layer deposition, or protein-enabled deposition).

The titanium compound-bearing coating may then undergo a low-temperature reduction reaction to yield a titanium composite coating. For example, a titania coating on a titanium-bearing body surface may undergo the following magnesiothermic reduction to yield a nanostructured MgO/Ti coating:

TiO₂(s)+2Mg(g,l,s)->2MgO(s)+Ti(s) (1)

In this example, magnesium (as a vapor, liquid, or solid) can be used as a reducing agent to convert titania into elemental titanium. Thermodynamic calculations indicate that this reaction is strongly preferred, even at modest temperatures. For example, the standard Gibbs free energy change per mole of this reaction, ΔG°ᵣₓₙ, is −304.2 kJ/mol at 400° C. At 900° C., ΔG°ᵣₓₙ remains quite negative at −280.1 kJ/mol. These highly negative values of ΔG°ᵣₓₙ, which indicate a much stronger affinity of oxygen for magnesium than for titanium, also indicate that the concentration of residual oxidized titanium is likely to be quite low in the presence of excess magnesium. Furthermore, the grain growth of the titanium product phase will likely be inhibited by the presence of the neighboring magnesia product phase. Hence, the magnesiothermic reduction of titania at a modest temperature can yield titanium domains/crystals of quite fine size.

This magnesiothermic reaction (eq. 1) yields products comprising about 67.9 vol % MgO and about 32.1 vol % Ti. If these products are generated as a random and uniform mixture of phases, then both product phases can form as interconnected networks (since both phase contents are above the expected percolation value for 3-D interconnectivity). Furthermore, selective dissolution of either phase then yields a porous, but interconnected network of the other phase. Since MgO is abasic oxide, selective acid dissolution of this phase would yield a highly porous (about 67.9 vol % porosity) network of interconnected titanium (assuming that the MgO and Ti products formed as a random and uniform mixture of phases).

As another example, a titania coating on a titanium-bearing body surface may undergo calciothermic reduction to yield a nanostructured CaO/Ti coating:

TiO₂(s)+2Ca(g,l,s)->2CaO(s)+Ti(s) (2)

As for the magnesiothermic reduction of titania, the calciothermic reduction of titania is also highly favored from a thermodynamic perspective. For example, at 400° C., the standard Gibbs free energy change per mole of this reaction, ΔG°ᵣₓₙ, is −242.4 kJ/mol (at 900° C., ΔG°ᵣₓₙ remains quite negative at −227.5 kJ/mol). These highly negative values of ΔG°ᵣₓₙ, which indicate a much stronger affinity of oxygen for calcium than for titanium, also indicate that the concentration of residual oxidized titanium is likely to be quite low in the presence of excess calcium. Furthermore, the grain growth of the titanium product phase will likely be inhibited by the presence of the neighboring calcia product phase. Hence, the calciothermic reduction of titania at a modest temperature can yield titanium domains/crystals of quite fine size.

This calciothermic reaction (eq. 2) yields products comprising about 75.8 vol % CaO and about 24.2 vol % Ti. Hence, if these products are generated as a random and uniform mixture of phases, then both product phases could form as interconnected networks (if both phase contents are above the percolation value for 3-D interconnectivity). Furthermore, selective dissolution of either phase would yield a porous, but interconnected network of the other phase. Since CaO is a basic oxide, selective acid dissolution of this phase would yield a highly porous (about 75.8 vol % porosity) network of interconnected titanium (assuming that the CaO and Ti products formed as a random and uniform mixture of phases).

The extent of porosity in the porous metallic titanium generated by this process may be tailored via the choice of the reducing agent. For Ca as a reducing agent, porous metallic titanium coatings with about 75.8 vol % porosity may be produced (assuming that the CaO and Ti products formed as a random and uniform mixture of phases). With Mg as the reducing agent, porous metallic titanium coatings with about 67.9 vol % porosity may be produced (assuming that the MgO and Ti products formed as a random and uniform mixture of phases). The porosity may be further tailored by starting with a titanium-bearing compound containing other constituents that may be dissolved away. For example, if a Ca₃Ti₂O₇ coating were applied to a Ti-bearing body, the titanium cations in this oxide can be selectively reduced to titanium via the calciothermic reduction reaction:

Ca₃Ti₂O₇(s)+4Ca(g,l,s)->7CaO(s)+2Ti(s) (3)

The resulting CaO/Ti composite contains about 84.6 vol % CaO and about 15.4 vol % Ti. If the metallic titanium in such a composite is interconnected, then selective dissolution of the CaO (e.g., via acid dissolution) would yield porous metallic titanium with about 84.6 vol % porosity (assuming that the CaO and Ti products formed as a random and uniform mixture of phases).

In addition to tailoring the pore fraction in the resulting porous titanium films on titanium-based bodies (for example, for biomedical implants), the methods could be used to achieve titanium-bearing surfaces with independently-tailored roughness at both the nanoscale and microscale. For example, the low-temperature reduction of titania coatings into MgO/Ti and CaO/Ti composite coatings can yield surfaces with enhanced nanoscale roughness compared to the starting titania coating. Subsequent selective dissolution of the oxide phase, yields nanoscale pores that further add to the nanoscale roughness of the porous metallic titanium film.

The conformal, shape-preserving nature of the low-temperature reduction processes of these methods allows the microscale surface topography of the starting Ti-bearing body to be preserved in the oxide/Ti and porous metallic titanium-bearing films. Hence, by providing a microscale roughness to the Ti-bearing body surface, the shape-preserving nature of the methods may add nanoscale roughness while retaining the applied microscale roughness. Microscale roughness may be applied to the initial body by a variety of methods including, but not limited to, chemical etching.

CaO/Ti films or MgO/Ti films formed on Ti-bearing surfaces via calciothermic or magnesiothermic reaction, respectively, with titania present on the Ti-bearing body surface can also be directly utilized for certain applications, such as biomedical implants. Such oxide/titanium coatings, particularly CaO/Ti coatings, are likely to promote bone growth by providing a local source of some of the constituents present in biological hydroxyapatite (the major mineral in human teeth and bones). Gradual resorption of such oxide constituents in the oxide/titanium films will also gradually provide additional porosity and surface roughness during osseointegration of a biomedical implant.

In summary, the methods herein may be used to modify at least four surface characteristics of titanium-bearing surfaces and bodies, such as those of biomedical implants, including roughness, porosity, grain size, and composition. For biomedical implant applications, surface roughness is a critical factor for cell behavior and implant success. Several studies have shown that sub-micron and nano-scale roughness also have a marked effect on cell behavior, especially when combined with macro-roughness. Similarly, porosity positively affects osseointegration both on a macroscale and sub-micron scale, providing micromechanical interlocking and preferential cell attachment. The grain size of titanium has also been shown to affect osseointegration, with ultra-fine or nanocrystalline titanium showing improved response.

Nonlimiting embodiments of the invention will now be described in reference to experimental investigations associated with the invention.

To demonstrate the feasibility of the proposed methods for generating porous Ti-bearing surfaces on Ti-bearing substrates, magnesiothermic and calciothermic reaction processes were applied to TiO₂(s) in the form of powder (to generate MgO/Ti composite powder) and in the form of an oxide layer generated on titanium-bearing substrates (to generate conformal MgO/Ti and CaO/Ti films, and porous Ti-bearing films, on Ti-bearing substrates), as described in the following examples.

### Example #1: Magnesiothermic Reduction of Titania (TiO₂) Powder into Nanocrystalline Ti/MgO Composite Powder

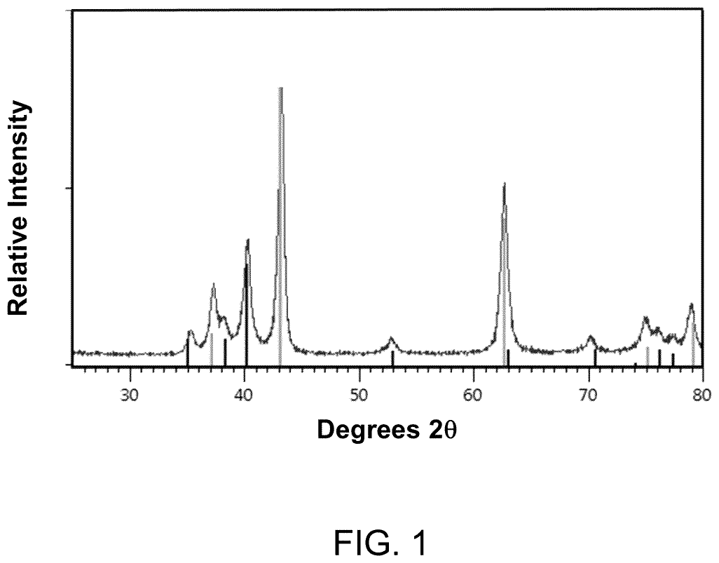

According to a preferred but nonlimiting embodiment, magnesium vapor, generated by evaporation from liquid magnesium, was used as a reducing agent. For the case of titania powder, rutile titania particles, with sizes in the range of 0.9 to 1.6 μm, were allowed to react with the Mg vapor within a sealed metal ampoule at 700° C. for 12 hours and at 900° C. for 12 hours (note: the melting point of magnesium is 650° C.). After reaction at 700° C. for 12 hours, X-ray diffraction (XRD) analyses (FIG. 1) confirmed the presence of only MgO and Ti product phases. Scherrer analysis of the diffraction peaks revealed average crystal sizes of 73 nm for the MgO product and 32 nm for the metallic titanium product. After reaction at 900° C. for 12 hours, XRD analyses again confirmed the presence of only MgO and Ti, with average crystal sizes of 156 nm and 206 nm for MgO and Ti, respectively. These experiments confirmed that titania can be completely converted into a mixture of MgO and Ti phases with crystal sizes that can be adjusted with the reaction conditions, while still remaining in the size range of a few hundred nm or less.

### Example #2: Magnesiothermic Reduction of Titania Films Present on Partially-Oxidized Titanium Substrates to Yield Conformal MgO/Ti Films, and Porous Ti Films, of Tailorable Thickness on Titanium Substrates

This example demonstrates that a non-line-of-sight reaction process involving a magnesiothermic reaction may also be used to generate thin conformal films comprising nanocrystalline MgO/Ti, or porous nanocrystalline Ti, on Ti-based substrates. Such nanocrystalline MgO/Ti-coated or porous Ti-coated surfaces are attractive for Ti-based biomedical implants, given reports of the enhanced interactions of bone-forming cells on nanostructured Ti-based surfaces.

Titanium (Ti) disks (15 mm diameter) were punched from grade 2 unalloyed Ti sheet (1 mm thick, ASTM F67 unalloyed Ti for surgical implant application, 0.30 weight % Fe, 0.25 weight % O, 0.08 weight % C, 0.015 weight % H, 0.03 weight % N, 4.51 g/cm³, 95% relative density) and supplied by Institut Straumann AG (Basel, Switzerland). The disks were ground and polished using a series of silicon carbide abrasive paper (320, 600, 800, 1200 ANSI grit, Allied High Tech Products, Inc. Rancho Dominguez, Calif., USA), diamond polycrystalline paste (1 μm, Allied High Tech Products, Inc.), and colloidal silica (0.05 μm, Allied High Tech Products, Inc.). A METPREP 3 Grinder/Polisher (Allied High Tech Products, Inc.) was used for the manual grinding and polishing. The disks were then ultrasonically cleaned in acetone (99.5% purity, Fisher, Hampton, N.H., USA) and ethanol (99.5% purity, Fisher) for 10 min each at room temperature.

Polished Ti disks were placed in a rectangular alumina crucible (107 mm×30 mm×15 mm, 99.8% purity, Almath Crucibles Ltd, UK) and then heated at 5° C./min in stagnant ambient air (10% relative humidity at 18-22° C.) to 600° C. and held at this temperature for 6 h to allow for partial oxidation. The oxidized Ti disks were then allowed to react with magnesium vapor within sealed metal ampoules. A given oxidized disk was placed in a small rectangular crucible (20 mm×20 mm×5 mm) made of titanium foil (>99.9% purity, 0.1 mm thick, MTI Corporation, USA). Titanium foil crucibles of similar dimensions containing magnesium granules (0.02±0.01 g, 99.8% purity, 0.30 mm≤granule size <1.7 mm, Beantown Chemical, Hudson, N.H., USA) were also prepared. These granules were stored in argon atmosphere glovebox (≤3.0 ppm O₂, ≤3.0 ppm H₂O). It was confirmed by X-ray diffraction (XRD) analysis that these granules had not formed a detectable thickness of oxide layer before the experiments. Two oxidized-disk-bearing titanium crucibles were placed, in an alternating manner, with three magnesium-granule-bearing titanium crucibles inside a low-carbon steel tube (3.6 cm internal diameter×29.2 cm long×1.2 mm thick, 1008/1010 steel, McMaster-Carr, Elmhurst, Ill., USA). Both ends of the tube were crimped shut and then sealed by TIG welding (Miller Electric Manufacturing Co., Appleton, Wis., USA) within an argon atmosphere glove box (≤3.0 ppm O₂, ≤3.0 ppm H₂O). The specimen-bearing steel ampoule was then heated in a flowing (154 ml/min) high-purity argon (99.999% purity, 1 ppm O₂, Airgas, Radnor, Pa., USA) atmosphere (to minimize oxidation of the steel) at 5° C./min to 700° C. and held at this temperature for 10 min to 1 h (to allow for the generation of Mg vapor, and reaction of this vapor with the oxidized specimens), followed by cooling at 5° C./min to 25° C. After the removal from the steel ampoule, the disks were statically immersed in 40 ml of 3 M acetic acid solution (manually diluted from 99.5% acetic acid from Fisher) for 40 min at room temperature to selectively dissolve the MgO from the reacted specimens.

Images of specimen surfaces at various stages of reaction were obtained using scanning electron microscopy (Through the Lens Detector, TLD, for high magnification secondary electron images: NOVA FE-SEM, FEI Co., Hillsboro, Oreg., USA; Everhart Thornley Detector, ETD, for low magnification secondary electron images: Quanta 3D FEG-SEM, FEI Co.). Scanning electron microscope (SEM) images were acquired using an acceleration voltage and current of 5 kV and 0.11 nA for NOVA and 10 kV and 4.0 nA for Quanta, respectively. Energy dispersive X-ray (EDX) analyses for elemental mapping were acquired using an Oxford INCA Xstream-2 silicon drift detector with an Xmax80 window with an acceleration voltage and current of 10 kV and 16-48 nA, respectively. Specimen cross-sections were prepared by focused ion beam milling (Quanta 3D FEG-SEM, FEI Co.) at 30 kV and 30 pA after deposition of a top Pt layer at 20 kV and 0.3 nA. Specimen cross-sections were transferred to a copper grid to allow for transmission electron microscopy (Tabs 200X, FEI Co.). Elemental maps of such cross-sections were acquired under STEM (scanning transmission electron microscopy) mode with EDX analyses. The thickness measurements by image analyses were conducted using ImageJ, by measuring thicknesses at 30 locations in the cross-sectional STEM image or the associated elemental map. Atomic force microscopy (AFM, NanoScope IIIa, Digital Instrument, Veeco Metrology Group, Santa Barbara, Calif., USA), with an n-doped Si cantilever that possessed a resonance frequency of 300 kHz (Bruker, Billerica, Mass., USA), was used to evaluate the nanoscale surface roughness. The scan rate was 0.2 to 0.5 Hz with 512 lines.

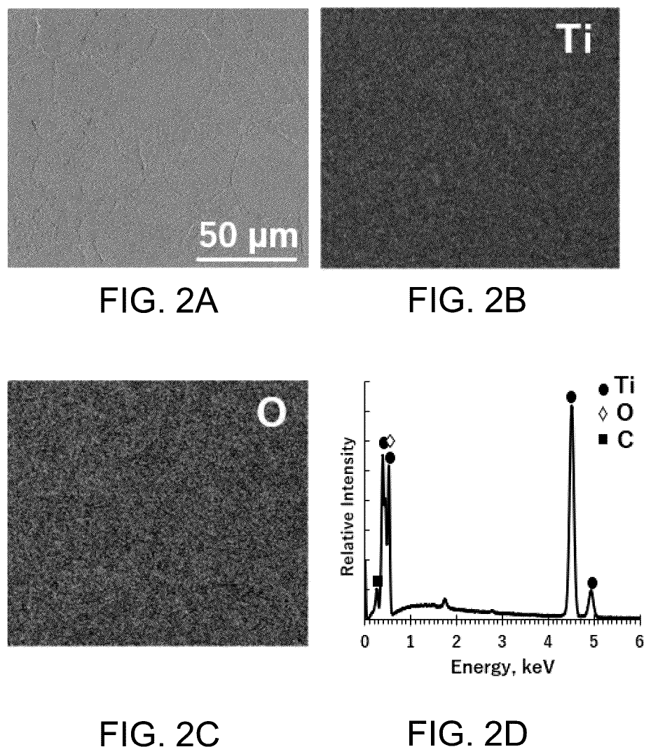

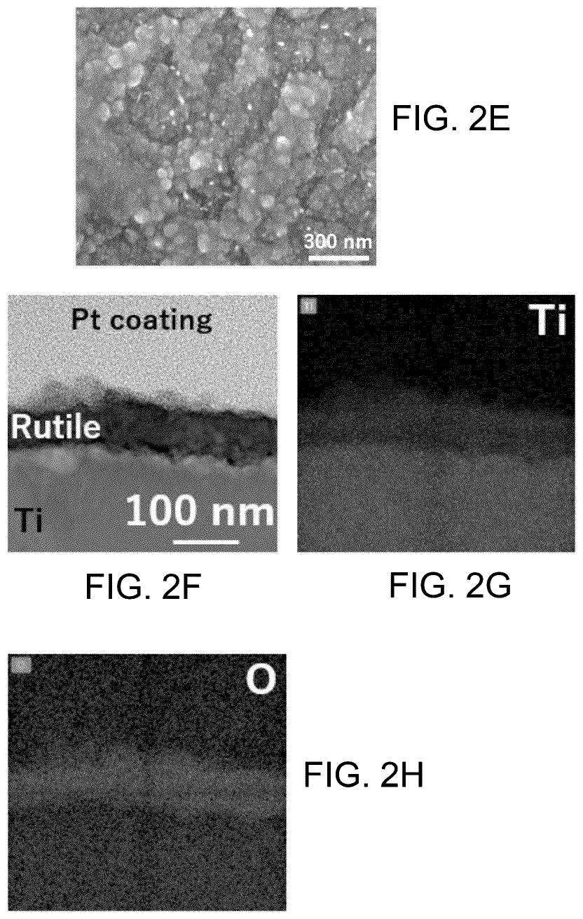

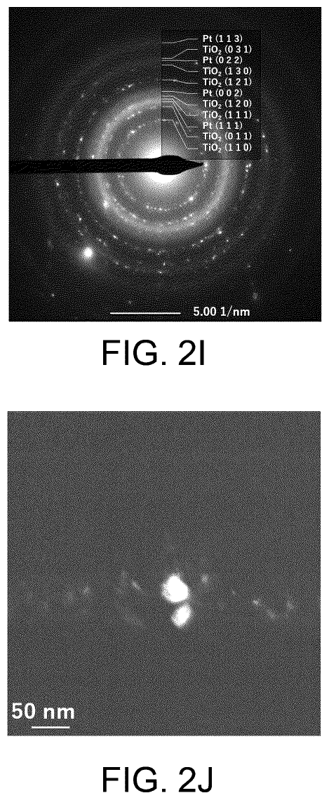

After oxidation in air for 6 h at 600° C., the Ti was covered with a continuous oxide scale, as confirmed by EDX analysis (e.g., from the uniform Ti and O maps in FIGS. 2B and 2C, respectively, associated with the secondary electron (SE) image in FIG. 2A; from the enrichment of oxygen in FIG. 2D relative to the EDX analysis of the starting unoxidized Ti sample). A high-resolution SE image of the external surface of this oxidized Ti specimen, and a STEM image of a focus-ion-beam milled cross-section of the oxidized Ti specimen, revealing a continuous, nanocrystalline external oxide scale, are shown in FIG. 2E and FIG. 2F, respectively. The average thickness of the oxide scale upon oxidation at 600° C. was 60±4 nm. The oxide scale comprised the rutile polymorph of TiO₂, as confirmed by the cross-sectional selected area electron diffraction (SAED) analysis and elemental maps (FIGS. 2G, 2H, and 2I). A dark field TEM image obtained from the rutile scale (FIG. 2J) revealed rutile crystals of 30-50 nm in size.

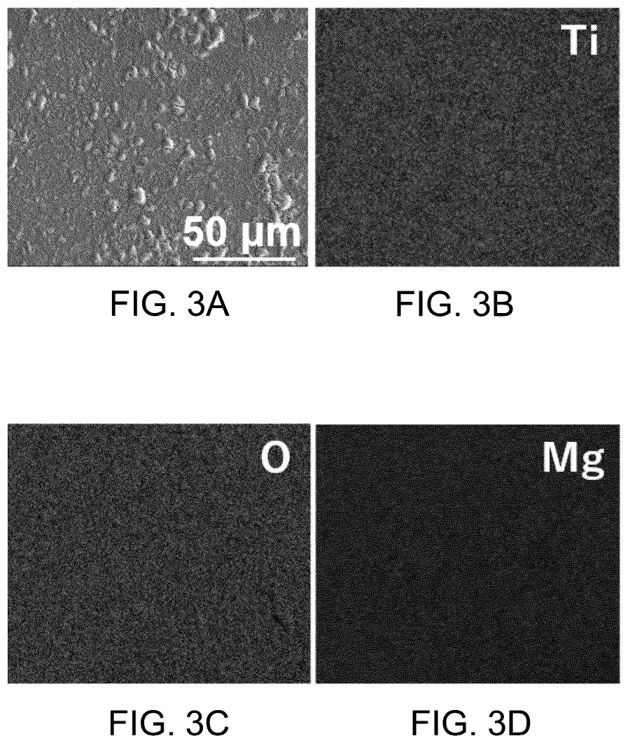

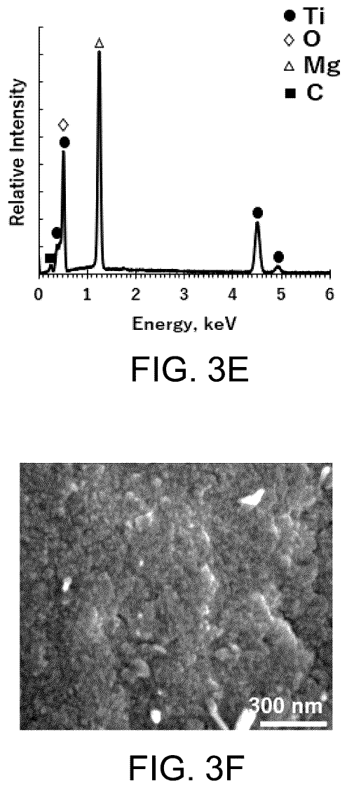

After selective reaction with Mg(g) for 1 h at 700° C., the external TiO₂ scale was converted into MgO and Ti products, as confirmed by EDX analysis (e.g., from the uniform Ti, O, and Mg maps in FIGS. 3B, 3C, and 3D, respectively, associated with the secondary electron (SE) image in FIG. 3A; from the enrichment of magnesium in FIG. 3E relative to FIG. 2D). The nanorough nature of the external surface of this specimen is seen in the high-magnification SE image in FIG. 3F.

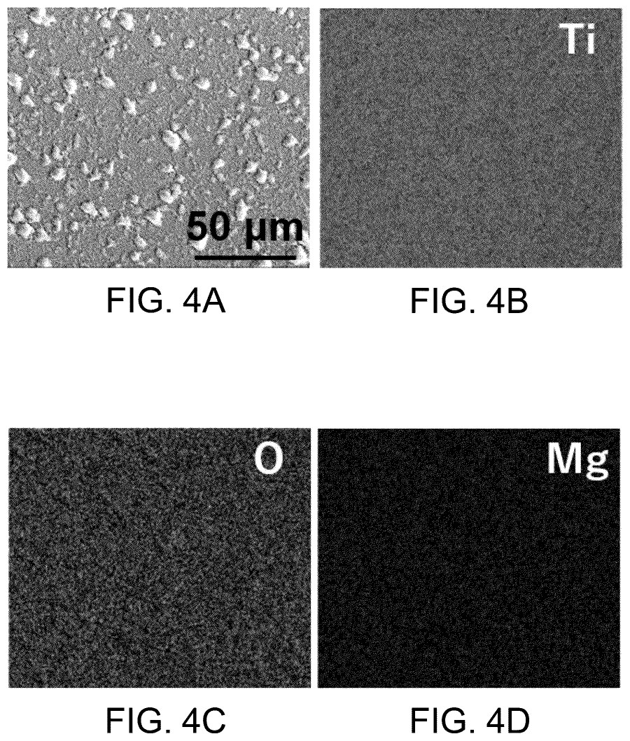

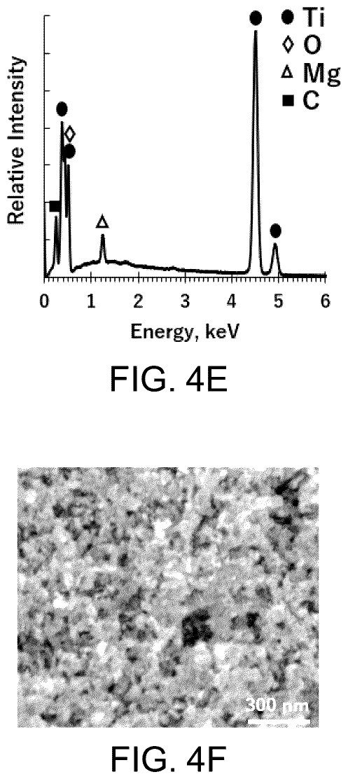

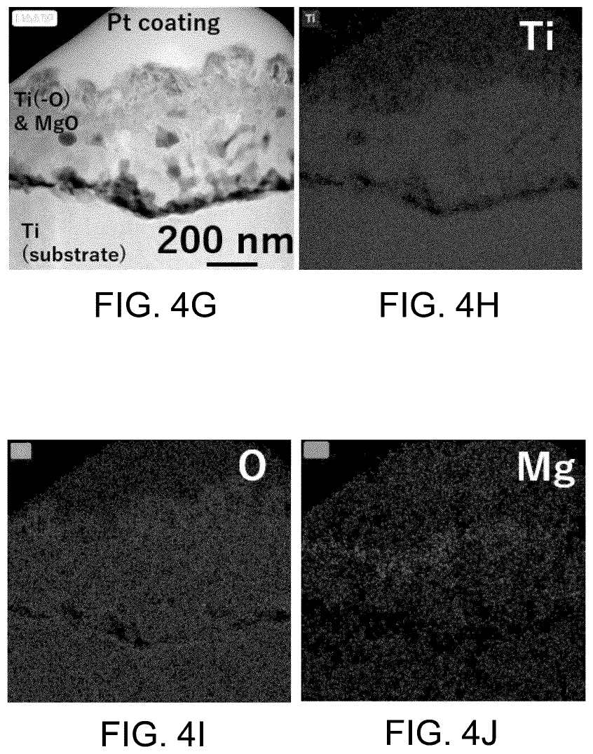

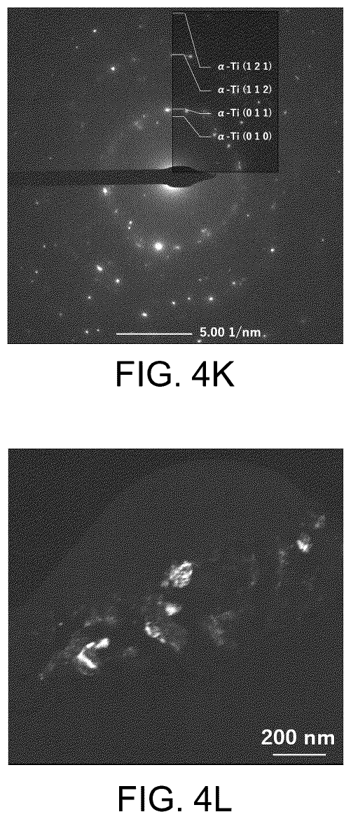

After selective dissolution of the MgO product phase in acetic acid, the specimen surfaces were converted into a nanoporous Ti-rich phase, as confirmed by EDX analysis (e.g., from the uniform Ti map in FIG. 4B, and reduced O and Mg in the maps in FIGS. 3C and 3D, respectively, associated with the secondary electron (SE) image in FIG. 4A; from the decrease of magnesium and oxygen intensities in FIG. 4E relative to FIG. 3E). A high-resolution SE image of the external surface of this specimen, and a STEM image with associated elemental maps of a focus-ion-beam milled cross-section of the Ti specimen after oxidation, magnesiothermic reaction, and MgO dissolution, shown in FIG. 4F and FIGS. 4G-4J, respectively, revealed a nanoporous Ti-rich layer with a thickness of 494±32 nm. The absence of MgO in the SAED pattern (FIG. 4K) suggests that the dissolution treatment was able to dissolve most of the MgO away. A dark field TEM image (FIG. 4L) revealed Ti crystals with sizes of 50-300 nm.

### Example #3: Magnesiothermic Reduction of Titania Films Present on Titanium Substrates to Yield Conformal MgO/Ti Films, and Porous Ti Films, on Titanium Substrates

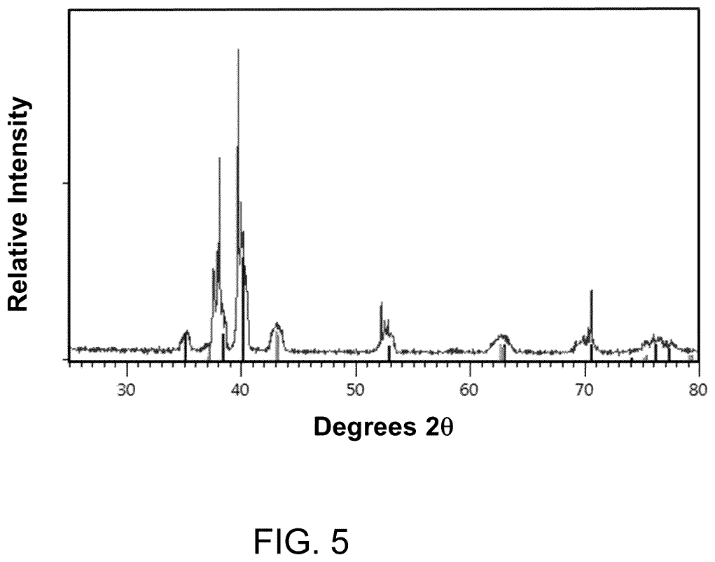

Titanium oxide (TiO₂) layers were first formed by the partial oxidation of commercially-pure metallic titanium disks at 740° C. for 1.5 hours in flowing air. The pre-oxidized disk was placed downstream from a magnesium source within a controlled atmosphere tube furnace. After heating both the disk and the magnesium to 700° C., flowing (oxygen-gettered) argon was then used to transport the Mg(g) generated from the magnesium source to the heated titania (TiO₂) coated metallic titanium disks to allow for selective magnesiothermic reaction of the titania coating with Mg(g). (Note: while the reaction of titania with Mg(g) is strongly favored, as mentioned above, metallic titanium is chemically compatible with Mg and, hence, will not react with Mg to form new compounds.) After reaction with Mg(g) for 24 hours, the specimens were examined with grazing incidence XRD analysis (FIG. 5). The presence of MgO and Ti diffraction peaks, and the absence of titania diffraction peaks, confirmed that the titania coating was completely converted into MgO and Ti.

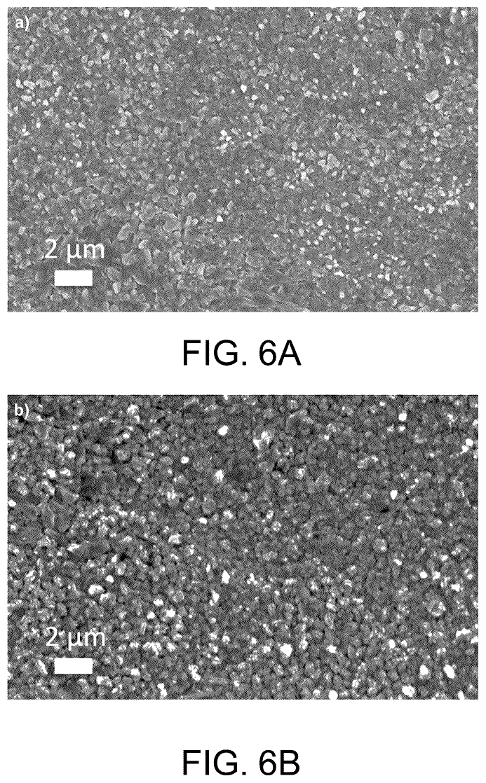

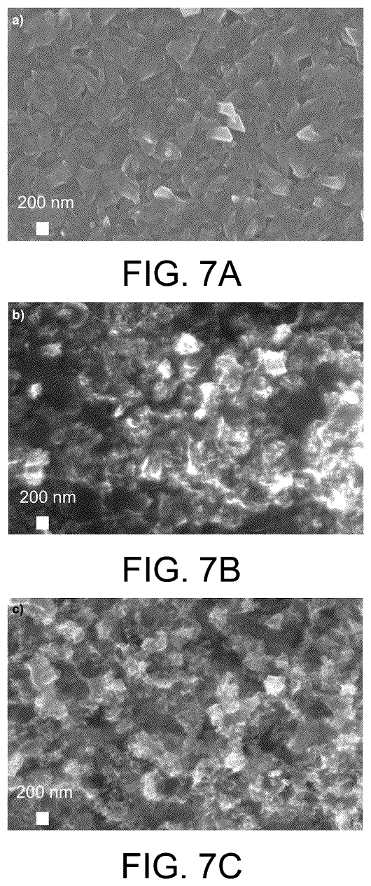

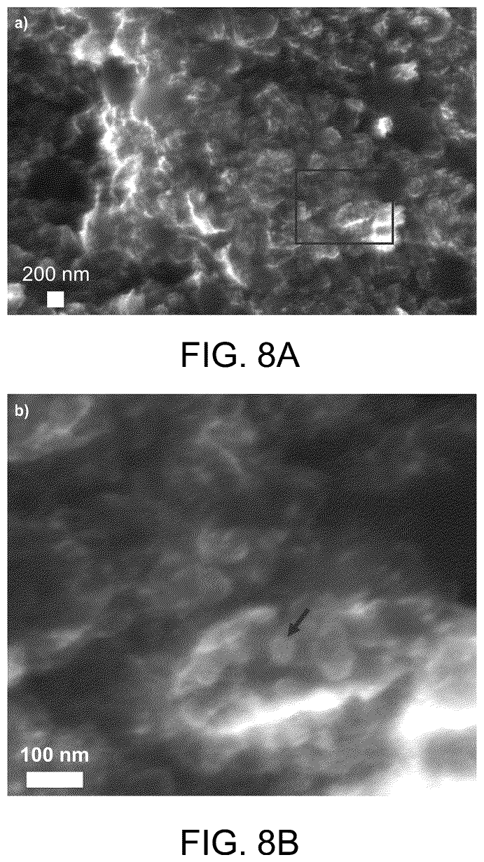

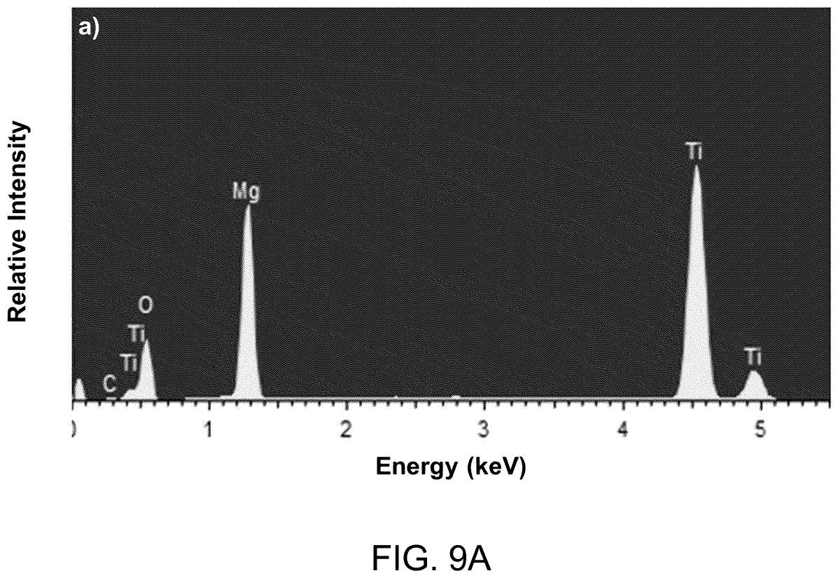

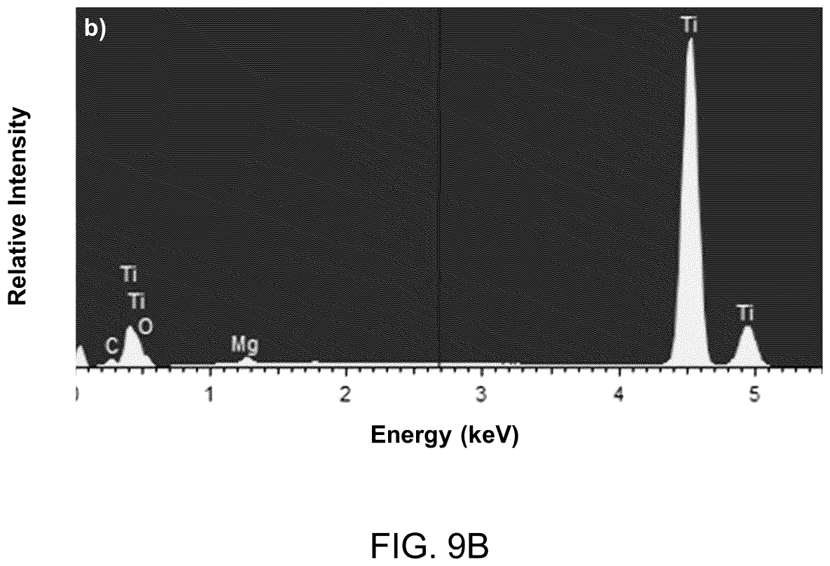

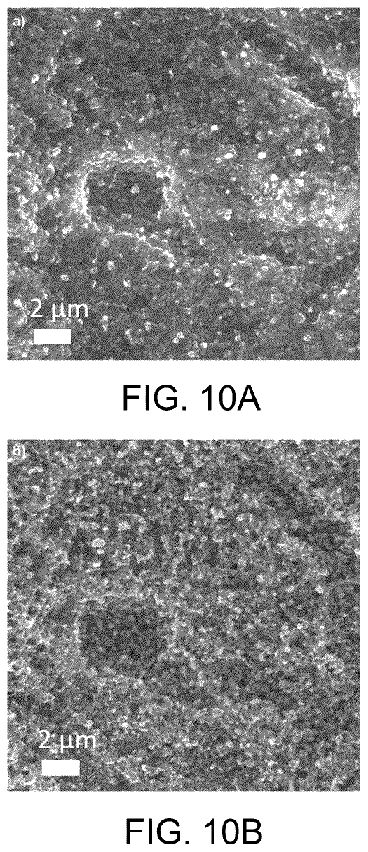

Scanning electron microscopy was then conducted to evaluate the change in morphology of the partially-oxidized titanium disk surface before and after such reaction. As revealed in the low- and high-magnification, top-down secondary electron images in FIGS. 6A and 7A, respectively, the titania crystals on the as-oxidized surface (740° C./1.5 hours/air) of the metallic titanium disk possessed sizes on the order of several hundred nm. After conversion of the titania film into a MgO/Ti composite film, the surface appeared to be noticeably rougher at the nanoscale (FIGS. 6B, 7B, 8A, and 8B). High magnification images (FIGS. 7B, 8A, and 8B) revealed the presence of very fine nanoparticles (a few tens of nm in size) on top of larger particles (a few hundred nm in size) on the surface of the MgO/Ti composite film. The MgO product phase was then selectively removed from the MgO/Ti film via exposure to 1 M HCl for 3 hours (as confirmed by energy dispersive X-ray analyses before and after HCl exposure in FIGS. 9A and 9B, respectively). After such selective dissolution of the MgO product phase from the film, fine pores (a few hundred nm in diameter) were generated (FIG. 10B). As discussed in the first example above, the average size of the MgO crystals generated by magnesiothermic reaction with titania at 700° C. was 110 nm. Hence, the diameters of pores generated by selective MgO dissolution (FIG. 10B) corresponded to domains of a few MgO crystals in size. The enhanced nanoscale roughness, associated with the very fine metallic titanium nanoparticles and the fine pores, in the porous metallic titanium layer was achieved while retaining the microscale features/roughness of the MgO/Ti layer (compare FIGS. 10A and 10B).

### Example #4: Calciothermic Reduction of Titania Films Present on Partially-oxidized Titanium Substrates to Yield Conformal CaO/Ti Films, and Porous Ti Films, of Tailorable Thickness on Titanium Substrates

Titanium (Ti) disks (15 mm diameter) were punched from grade 2 unalloyed Ti sheet (1 mm thick, ASTM F67 unalloyed Ti for surgical implant application, 0.30 weight % Fe, 0.25 weight % O, 0.08 weight % C, 0.015 weight % H, 0.03 weight % N, 4.51 g/cm³, 95% relative density) and supplied by Institut Straumann AG (Basel, Switzerland). The disks were ground and polished using a series of silicon carbide abrasive paper (320, 600, 800, 1200 ANSI grit, Allied High Tech Products, Inc. Rancho Dominguez, Calif., USA), diamond polycrystalline paste (1 μm, Allied High Tech Products, Inc.), and colloidal silica (0.05 μm, Allied High Tech Products, Inc.). A METPREP 3 Grinder/Polisher (Allied High Tech Products, Inc.) was used for the manual grinding and polishing. The disks were then ultrasonically cleaned in acetone (99.5% purity, Fisher, Hampton, N.H., USA) and ethanol (99.5% purity, Fisher) for 10 min each at room temperature.

Polished Ti disks were placed in a rectangular alumina crucible (107 mm×30 mm×15 mm, 99.8% purity, Almath Crucibles Ltd, UK) and then heated at 5° C./min in stagnant ambient air (10% relative humidity at 18-22° C.) to 600° C. and held at this temperature for 6 h to allow for partial oxidation. The oxidized Ti disks were then allowed to react with calcium vapor within sealed metal ampoules. A given oxidized disk was placed in a small rectangular crucible (20 mm×20 mm×5 mm) made of titanium foil (≥99.9% purity, 0.1 mm thick, MTI Corporation, USA). Titanium foil crucibles of similar dimensions containing calcium granules (1.0±0.01 g, 99.5% purity, granule size <3.4 mm, Fisher, USA) were also prepared. These granules were stored in argon atmosphere glovebox (≤3.0 ppm O₂, ≤3.0 ppm H₂O). It was confirmed that these granules had not formed a detectable thickness of oxide layer before the experiments. Two oxidized-disk-bearing titanium crucibles were placed, in an alternating manner, with three calcium-granule-bearing titanium crucibles inside a low-carbon steel tube (3.6 cm internal diameter×29.2 cm long×1.2 mm thick, 1008/1010 steel, McMaster-Carr, Elmhurst, Ill., USA). Both ends of the tube were crimped shut and then sealed by TIG welding (Miller Electric Manufacturing Co., Appleton, Wis., USA) within an argon atmosphere glove box (≤3.0 ppm O₂, ≤3.0 ppm H₂O). The specimen-bearing steel ampoule was then heated in a flowing (154 ml/min) high-purity argon (99.999% purity, 1 ppm O₂, Airgas, Radnor, Pa., USA) atmosphere (to minimize oxidation of the steel) at 5° C./min to 700° C. and held at this temperature for 10 min to 1 h (to allow for the generation of Ca vapor, and reaction of this vapor with the oxidized specimens), followed by cooling at 5° C./min to 25° C. After the removal from the steel ampoule, the disks were statically immersed in 40 ml of 3 M acetic acid solution (manually diluted from 99.5% acetic acid from Fisher) for 40 min at room temperature to selectively dissolve the CaO from the reacted specimens.

After oxidation in air for 6 h at 600° C., the Ti was covered with a continuous oxide scale, as confirmed by EDX analysis (e.g., enrichment of oxygen in FIG. 2D relative to the EDX analysis of the starting unoxidized Ti sample). A STEM image of a focus-ion-beam milled cross-section of the oxidized Ti specimen, revealing a continuous, nanocrystalline external oxide scale, is shown in FIG. 2F. The average thickness of the oxide scale upon oxidation at 600° C. was 60±4 nm. The oxide scale comprised the rutile polymorph of TiO₂, as confirmed by the cross-sectional selected area electron diffraction (SAED) analysis and elemental maps (FIGS. 2G, 2H, and 2I). A dark field TEM image obtained from the rutile scale (FIG. 2J) revealed rutile crystals of 30-50 nm in size.

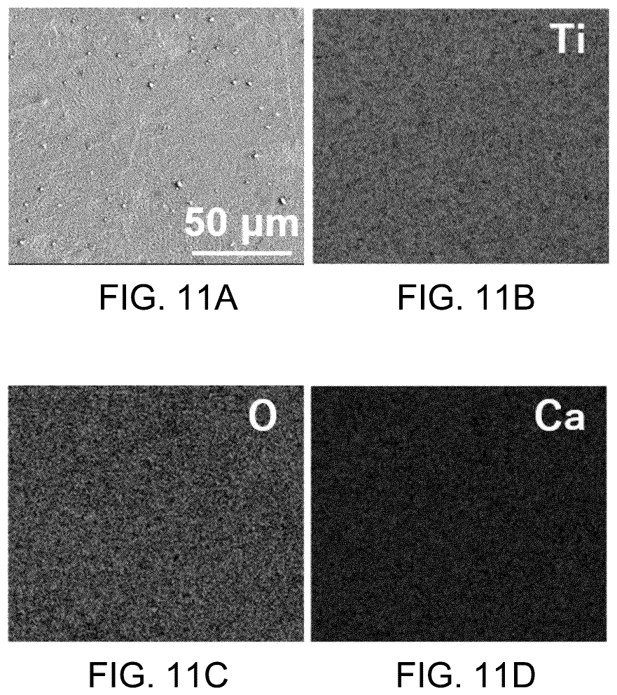

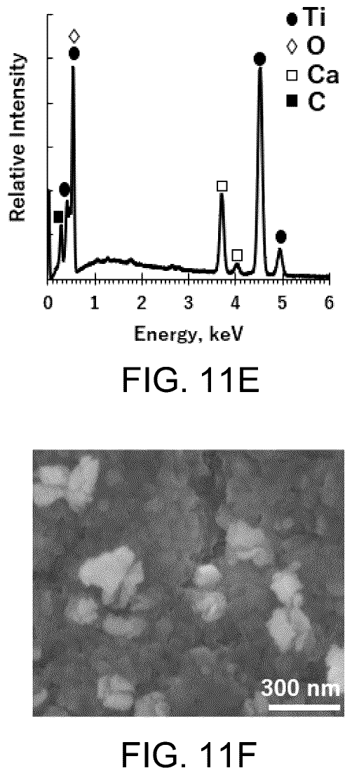

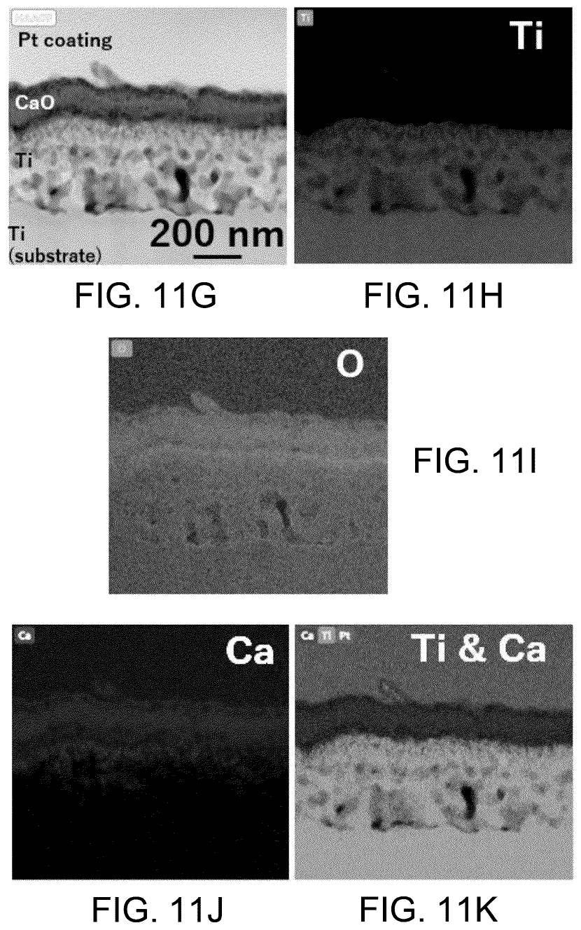

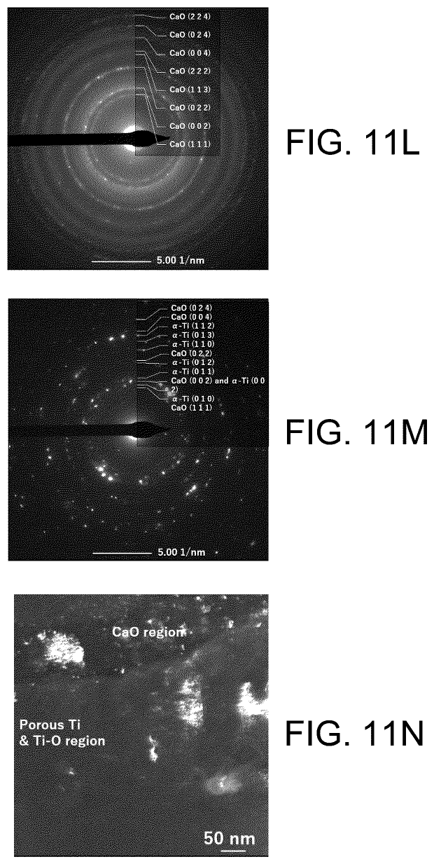

After thermal oxidation and selective reaction with Ca(g) for 1 h at 700° C., the external TiO₂ scale was converted into CaO and Ti products, as confirmed by EDX analysis (e.g., from the uniform maps for Ti, O, and Ca in FIGS. 11B-D associated with the SE image in FIG. 11A of the external surface of this specimen; from the enrichment of calcium in FIG. 11E relative to FIG. 2D). A high-resolution SE image of the external surface of this specimen, and a STEM image with elemental maps of a focus-ion-beam milled cross-section of the Ti specimen after oxidation and calciothermic reaction are shown in FIG. 11F and FIGS. 11G-11K, respectively. SAED analyses (FIGS. 11L, 11M) confirmed the presence of the CaO and Ti product phases and the absence of rutile TiO₂ after reaction of the oxidized specimen with Ca(g) for 1 h at 700° C. The thickness of the Ca-rich region was 298±21 nm, the thickness of the Ti-rich porous region was 316±32 nm, and the thickness of the entire product layers was 519±12 nm (from the TEM elemental maps and STEM image). A dark field TEM image (FIG. 11N) revealed CaO crystallite sizes smaller than 100 nm and Ti crystallite sizes of 60-150 nm

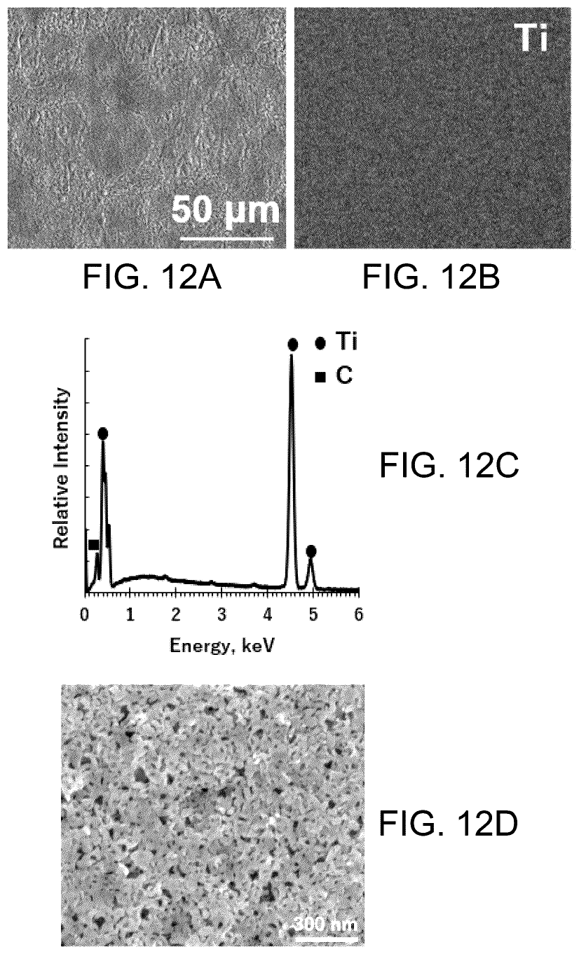

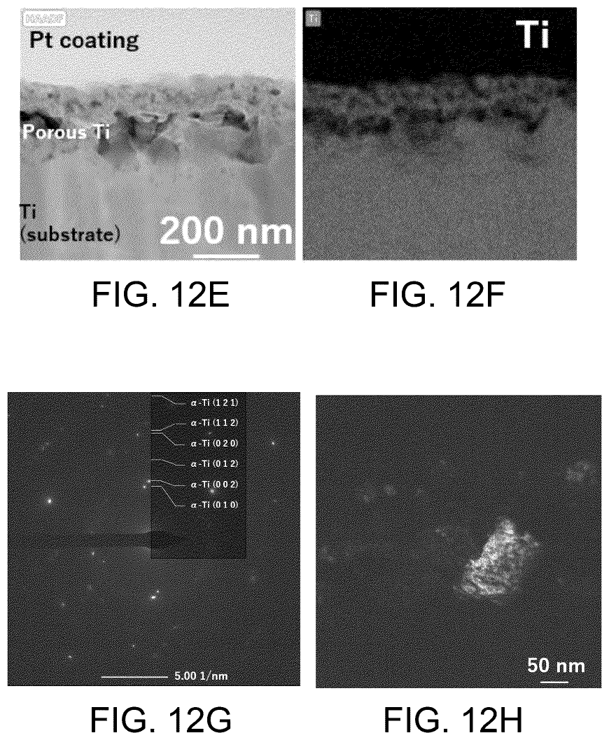

After selective dissolution of the CaO product phase in acetic acid, the specimen surfaces were converted into porous Ti, as confirmed by EDX analysis (i.e., from the uniform Ti map in FIG. 12B associated with the SE image of the external surface of this specimen in FIG. 12A; from the loss of calcium and oxygen in FIG. 12C relative to FIG. 11E). SAED analysis (FIG. 12G) confirmed the presence of the Ti product phase and the absence of CaO in this porous layer, which indicated that selective CaO dissolution was successful. A high-resolution SE image of the external surface of this specimen, and a STEM image with an associated Ti map of a focus-ion-beam milled cross-section of the Ti specimen after oxidation, calciothermic reaction, and CaO dissolution, shown in FIG. 12D and FIGS. 12E and 12F, respectively, revealed a nanoporous surface layer with a thickness of 258±15 nm. A dark field TEM image (FIG. 12H) revealed Ti crystals with sizes of 50-140 nm.

### Example #5: Calciothermic Reduction of Titania-based Films Present on Partially-Oxidized Titanium Alloy Substrates to Yield Conformal CaO/Ti-based Films, and Porous Ti-based Films, of Tailorable Thickness on Titanium Alloy Substrates

This example demonstrates that a non-line-of-sight reaction process involving a calciothermic reaction may be used to generate conformal films comprising nanocrystalline CaO/Ti, or porous nanocrystalline Ti, on Ti alloy substrates. Such nanocrystalline porous Ti-coated surfaces are also shown in this example to enhance osteoblastic responses of bone marrow stromal cells (MSCs) cultured on such reaction-modified surfaces.

Titanium-aluminum-vanadium alloy disks (15 mm diameter, 4 mm thick) were obtained from Virginia Commonwealth University. The disks were generated by direct metal laser sintering (DMLS) of plasma-atomized Ti—Al(6 weight %)-V(4 weight %) powder (grade 5 TiAl6V4, Advanced Powders & Coatings, Quebec, Canada). After atomization, the TiAl6V4 particles were sorted by sieving to the 25-45 μm diameter range (as per the ISO 13485 certification for medical devices). DMLS was conducted with a continuous 200 W ytterbium-doped fiber (1054 nm) laser system (EOS, EmbH Munchen, Germany) using a laser scanning speed of 7 m/s with a step size of 100 μm and a laser spot size of 0.1 mm. The disks were removed from the build plate by electro discharge machining. The average porosity of the disks was 4.2%, based on the Archimedes test using water as the buoyant fluid. The TiAl6V4 disk surfaces were grit blasted using calcium phosphate particles (AB Dental, Ashdod, Israel) and rinsed three times with ultra pure H₂O at room temperature. The disk surfaces were then etched with ultrasonication in a 0.3 N nitric acid solution for 5 min at 45° C., followed by rinsing two times (5 min each) in ultra-high purity water and then in 97% methanol for 5 min at 25° C. A pickling treatment was then conducted, which consisted of three 10 min ultrasonic rinses in ultrapure H₂O, followed by submersion in an 1:1 aqueous solution of 20 g/L NaOH and 20 g/L H₂O₂ for 30 min at 80° C., and ultrasonication in the ultrapure distilled H₂O for 10 min. The disk surfaces were then degreased in an industrial degreaser for 12 min, and then submerged in 65% aqueous nitric acid at 100° C. for 10 min, followed by rinsing three times in ultrapure distilled H₂O for 10 min. The surfaces were then blotted with lint-free laboratory wipes and dried in ambient air at 25° C. These grit blasted and etched disks are referred to herein as the “microrough” TiAl6V4 specimens. Samples were wrapped in aluminum foil and packaged in plastic bags before sterilization by gamma irradiation.

Some of the DMLS-sintered TiAl6V4 disks were ground and polished using a series of silicon carbide abrasive papers (320, 600 ANSI grit, Allied High Tech Products, Inc. Rancho Dominguez, Calif., USA), diamond polycrystalline pastes (9 μm to 3 μm, Allied High Tech Products, Inc.), and colloidal silica (0.05 μm, Allied High Tech Products, Inc.). A METPREP 3 Grinder/Polisher with PH-3 (Allied High Tech Products, Inc.) was used for the grinding and polishing. The disks were then ultrasonically cleaned in acetone and ethanol for 10 min each at 25° C. These polished disks are referred to herein as the “smooth” TiAl6V4 specimens.

The TiAl6V4 disks were placed in a rectangular alumina crucible (107 mm×30 mm×15 mm, 99.8% purity, Almath Crucibles ltd, UK) and then heated at 5° C./min in ambient air to 600° C. and held at this temperature for 6 h to allow for partial oxidation. The oxidized TiAl6V4 disks were then allowed to undergo reaction with calcium vapor within sealed metal ampoules. A given oxidized disk was placed in a small rectangular crucible (20 mm×20 mm×5 mm) made of titanium foil (>99.9% purity, 0.1 mm thick, MTI Corporation, USA). Titanium foil crucibles of similar dimensions containing calcium granules (1.0 g, 99.5% purity, ≤3.36 mm, Alfa Aesar, USA) were also prepared. Four oxidized-disk-bearing titanium crucibles were placed, in an alternating manner, with five calcium-granule-bearing titanium crucibles inside a low-carbon steel tube (3.6 cm internal diameter×29.2 cm long×1.2 mm thick, 1008/1010 steel, McMaster-Carr, Elmhurst, Ill., USA). Both ends of the tube were crimped shut and then sealed by welding within an argon atmosphere (≤3.0 ppm O₂, ≤3.0 ppm H₂O) glove box. The specimen-bearing steel ampoule was then heated in a flowing (154 ml/min) high-purity argon (99.999% purity, 1 ppm O₂, Airgas, Radnor, Pa., USA) atmosphere (to minimize oxidation of the steel) at 5° C./min to 700° C. and held at this temperature for 1 h (to allow for the generation of calcium vapor, and reaction of this vapor with the oxidized specimens), followed by cooling at 5° C./min to 25° C. After removal from the steel ampoule, the disks were immersed in 40 ml of 3 M acetic acid solution for 40 min at 25° C. to selectively dissolve the CaO from the reacted specimens. The disks were then ultrasonically cleaned for 10 min each in acetone, ethanol, and DI water (resistivity=18.2 MΩ at 25° C.). The samples were then dried under vacuum (−100 kPa) for 15 min at 25° C. Samples were stored in the argon glove box mentioned above, and were sealed inside the glove box with two plastic ziplock bags before shipment to Virginia Commonwealth University for cell studies.

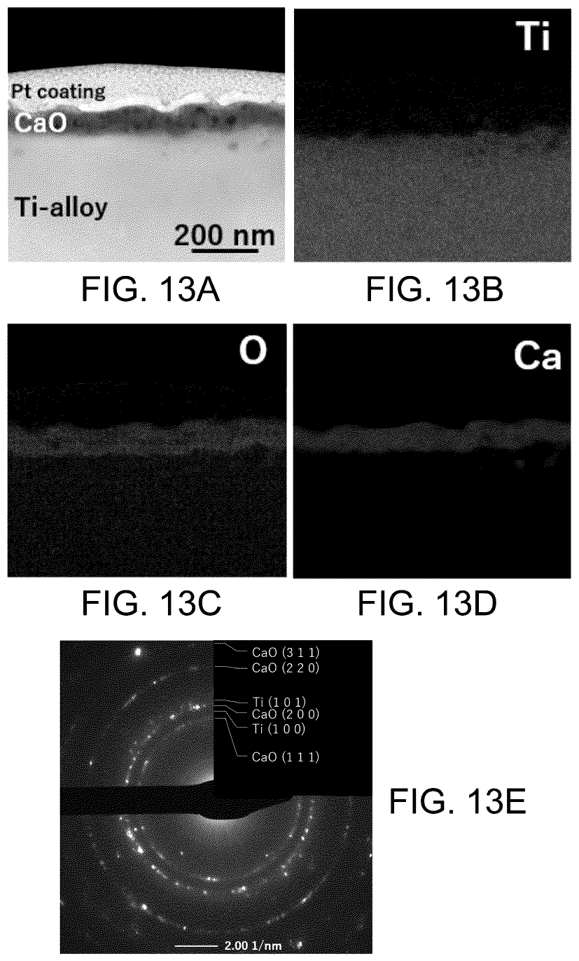

Although Ca(g) can react with titania (TiO₂) to form CaO/Ti films (eq. 2), this gas does not react further to form compounds with Ti. Consequently, this reaction yielded CaO/Ti films with thicknesses that were directly related to the thicknesses of the titania-based external scales formed upon oxidation. Hence, by controlling the oxidation conditions to form thin (submicron) titania-based scales on TiAl6V4 surfaces, the resulting CaO/Ti-bearing film was also submicron in thickness (as seen, for example, in the scanning transmission electron microscope image of an ion-milled cross-section in FIG. 13A). Elemental maps obtained by energy dispersive X-ray (EDX) analysis (FIGS. 13B-D) revealed the presence of Ti, O, and Ca in the product film seen in FIG. 13A, and selected area electron diffraction (SAED) analysis (FIG. 13E) indicated that this film comprised CaO and Ti.

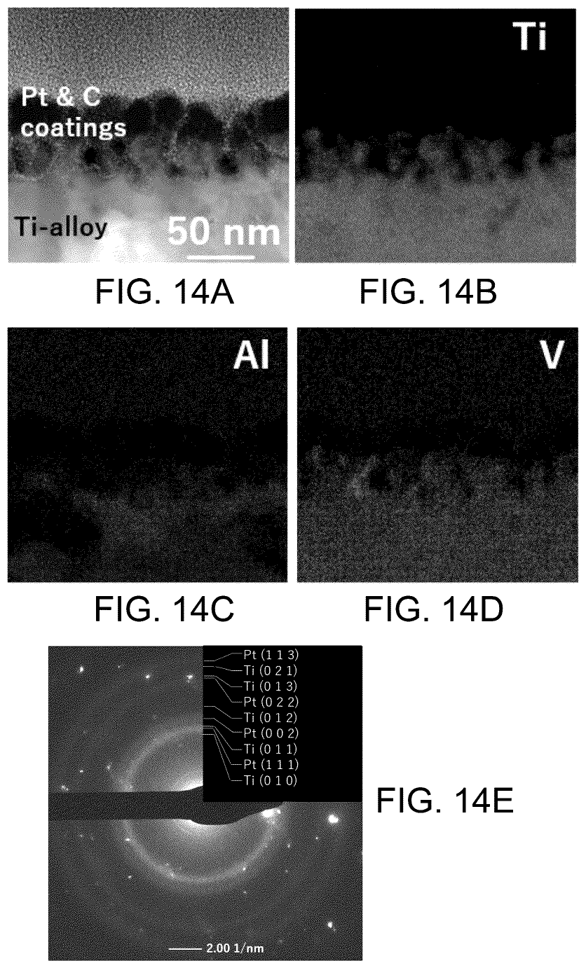

Selective CaO dissolution in the 3 M acetic acid solution then yielded thin (submicron) nanoporous metallic titanium-rich films that remained adherent to the dense (nonporous) TiAl6V4 alloy substrates after ultrasonic cleaning (as seen in the scanning transmission electron microscope image of an ion-milled cross-section in FIG. 14A). Elemental maps obtained by EDX analysis (FIGS. 14B-D) revealed the presence of Ti, Al, and V in the porous product film seen in FIG. 14A, and SAED analysis (FIG. 14E) indicated that this porous film comprised Ti (Pt was also detected, as Pt was deposited on the film to enhance the electrical conductivity of the film prior to ion milling).

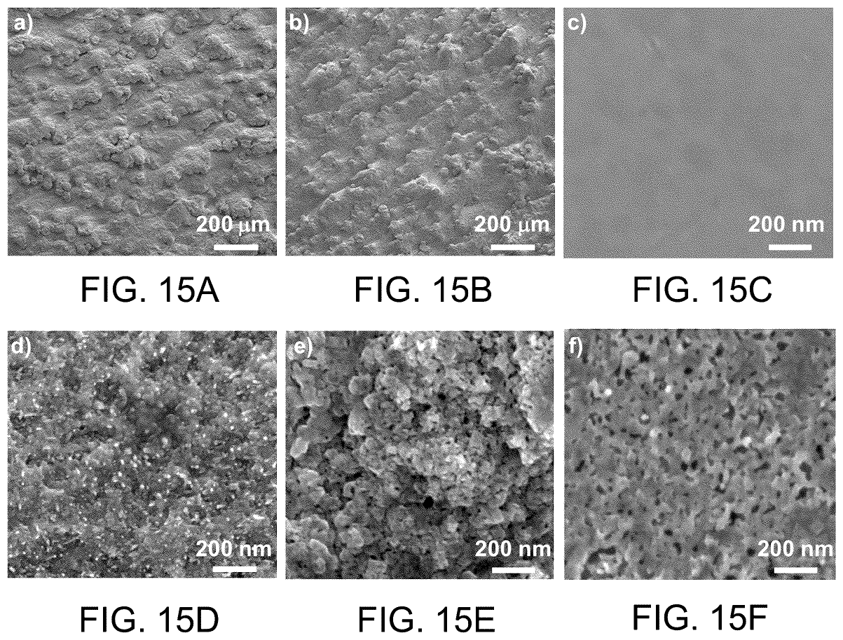

Secondary electron images of the external surfaces of laser-sintered TiAl6V4 alloys before and after such reaction processing in this example are shown in FIGS. 15A through 15F. Such processing resulted in the generation of surfaces with enhanced nanoscale roughness (as seen by comparing FIGS. 15D and 15E, and by comparing FIGS. 15C and 15F), while preserving the starting microscale roughness (compare FIGS. 15A and 15B). Atomic force microscopic (AFM) analyses of the TiAl6V4 alloys before and after such reaction processing also revealed a significant increase in the average nanoscale roughness (from 6±3 nm to 15±2 nm).

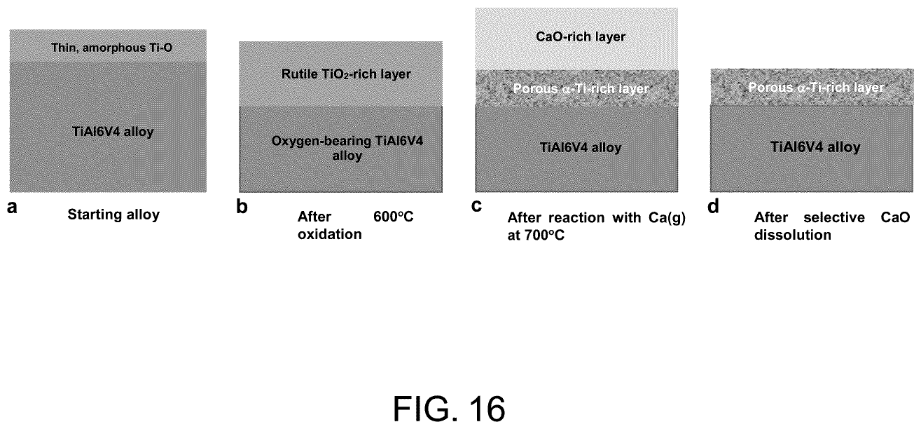

Top-down and cross-sectional secondary electron microscopy, energy-dispersive X-ray analyses, cross-sectional transmission electron microscopy, selective area electron diffraction analyses, and atomic force microscopic analyses, obtained at various stages of these reaction steps were found to be consistent with the surface morphological evolution illustrated in FIG. 16. After oxidation at 600° C. to form a thin (submicron), continuous, conformal, and adherent rutile titania surface layer, calciothermic reaction at 700° C. yielded a conformal product film comprising a CaO-rich external region and an underlying Ti-rich region. Selective CaO dissolution then yielded a thin (submicron) continuous, conformal, adherent, nanorough (nanoscale porous) metallic titanium surface layer on the dense (nonporous) metallic titanium alloy (TiAl6V4).

Human female bone marrow stromal cells (MSCs) donor #8011L (Texas A&M Institute for Regenerative Medicine, College Station, Tex.) were cultured by Virginia Commonwealth University in MSC growth medium (GM) comprised of αMEM with 4 μM L-glutamine and 16.5% fetal bovine serum at 37° C. in 5% CO₂ and 100% humidity and cultured to confluence in T75 flasks (Corning Inc., Oneonta, N.Y.) before plating on the surfaces. For biological analysis, surfaces were placed face down in the biological safety cabinet and sterilized by UV light for 24 h. The surfaces were then turned face up and again sterilized for another 24 h. The surfaces were then placed face up in 24-well plates, and cells were plated at a density of 20,000 cells/mL at 0.5 mL per well. MSCs cultured on tissue culture polystyrene (TCPS) served as experimental controls. The GM was changed 24 h after plating, with subsequent media changes conducted every subsequent 48 h for up to 7 days. At day 7, cells were incubated for 24 h with fresh GM before being harvested. Upon harvesting, conditioned media were subsequently collected and stored at −80° C., and MSCs were rinsed twice with 1×PBS, and placed in 0.5 mL of Triton-X100 and stored at −80° C. for biological assays. Cell layers were lysed by ultrasonication at 40 V for 15 sec/well (VCX 130; Vibra-Cell, Newtown, Conn.). The QuantiFluor*dsDNA system (Promega, Madison, Wis.) was used to determine total DNA content by fluorescence. Enzyme-linked immunosorbent assays were used to determine the levels of osteogenic factors in the conditioned media. Osteopontin (OPN; R&D Systems, Inc.) levels were quantified according to the manufacturer's protocol. Data were provided as means±standard error mean of six independent cultures/variable. All experiments were repeated to ensure the validity of observations, with results from individual experiments shown. Statistical analysis within a group was performed by one-way analysis of variance (ANOVA) and multiple comparisons between the groups were conducted with a two-tailed Tukey correction. A p-value of less than 0.05 was considered statistically significant. Such statistical analyses were performed with GraphPad Prism version 5.04.

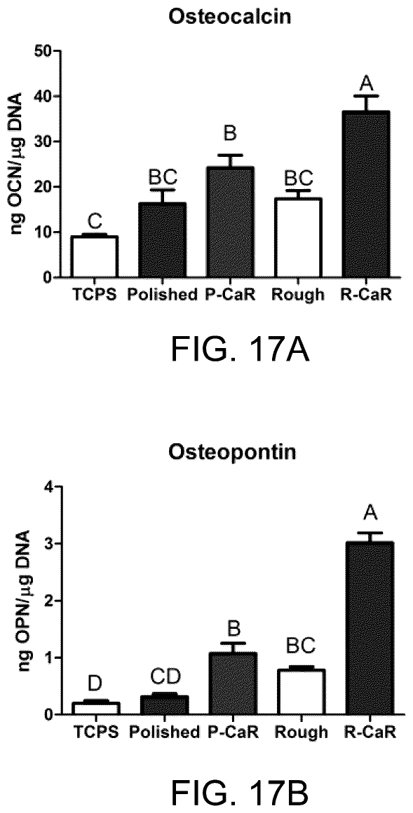

The MSCs cultured on TiAl6V4 alloy specimens that had been exposed to the calciothermic reduction process disclosed herein exhibited significant enhancements in cell behavior associated with bone formation. Relative to MSCs cultured on smooth (polished) or microrough (grit-blasted, acid etched) TiAl6V4 alloy specimens that were not exposed to the calciothermic reaction process, MSCs cultured on smooth or microrough TiAl6V4 alloy specimens exposed to the calciothermic reaction process exhibited significantly enhanced expression of osteocalcin (FIG. 17A), which is associated with osteoblast differentiation and maturation, and significantly enhanced expression of osteopontin (FIG. 17B), which is associated with bone formation.

The results described in these examples demonstrate that low-temperature (e.g., 700° C.) reduction of a titanium-bearing compound (e.g., titania) can be conducted with the use of a reducing agent (e.g., magnesium or calcium) contained within a fluid phase (e.g., a gas) to yield a nanocrystalline titanium-bearing composite (e.g., MgO/Ti, CaO/Ti) and, upon selective etching of the oxide product, nanocrystalline porous Ti. These results also demonstrate that the titanium-bearing compound (e.g., a titania film on a Ti-bearing substrate) can be converted into a Ti-bearing composite (e.g., a MgO/Ti film or CaO/Ti on the Ti-bearing substrate) and into porous metallic titanium (e.g., a porous metallic titanium film on the Ti-bearing substrate) that retains the shape of the starting titanium-bearing compound (e.g., a titania film). The average size of the pores in the final porous metallic titanium film, and the associated scale of the roughness of the film, can be tailored by the selection of the reducing agent (e.g., Mg or Ca) and by the conditions (e.g., temperature, time, partial pressure of the reducing agent) used for the reduction process.

Preferred initial bodies for use with the methods disclosed herein include bodies comprising titanium, a titanium alloy, and/or titanium oxide. A particularly suitable initial body for biomedical applications includes titanium-aluminum-vanadium (Ti—Al—V) alloys, such as the TiAl6V4 (90 weight % Ti, 6 weight % Al, 4 weight % V) alloy. In such embodiments, the product formed by the oxidation process preferably includes titanium oxide. The oxidation process preferably is performed at a temperature that is sufficiently high to achieve a desirable oxidation rate. As a nonlimiting example, suitable temperatures are believed to be within a range of about 100° C. to 900° C., for example, about 600° C. The reduction process preferably uses a reducing agent that includes an alkaline earth or an alkali element (such as magnesium, calcium, strontium, barium, lithium, sodium, potassium, and/or rubidium), preferably magnesium or calcium, and most preferably magnesium vapor or calcium vapor. The reduction process preferably produces a product that includes an oxide of an alkaline earth or an alkali element, most preferably magnesium oxide or calcium oxide. The reduction process also preferably produces a product that includes elemental titanium. The reduction process preferably is performed at a temperature that is sufficiently high to achieve a desirable reduction rate but not so high as to vaporize the reducing agent. As a nonlimiting example, suitable temperatures are believed to be within a range of about 100° C. to 900° C., for example, about 700° C. After completion of the selective dissolution process, an average diameter of the pores in the porous surface layer is preferably about 1 nm to about 300 nm, more preferably about 10 nm to about 300 nm, and most preferably about 10 nm to about 100 nm.

It should be understood that these reduction process may be applied to titanium compounds other than oxides (as a nonlimiting example, sulfides) and may involve the use of other reducing agents (as nonlimiting examples, strontium, barium, and lithium). The reducing agent may be present within a solid or fluid phase (with the fluid as a gas, liquid, or plasma). A wide variety of 1-dimensional, 2-dimensional, or 3-dimensional structures (including, but not limited to, particles, fibers, rods, tubes, films, plates, sheets, gears, honeycombs, crucibles, cones, dental implant shapes, maxillofacial implant shapes, orthopaedic implant shapes) containing a titanium compound may be partially or fully reduced by this process to yield a titanium-bearing composite or porous titanium-bearing structure that retains the shape of the starting titanium compound-bearing structure.

It is believed that a particularly beneficial application of these methods is to tailor the surfaces of biomedical implants to replace damaged/defective or lost bone-bearing structures in human or other animal bodies. Such structures include dental, maxillofacial, and orthopaedic implants. Currently, titanium and titanium alloys are commonly used materials for such implants. One of the critical concerns in the development and use of titanium-bearing biomedical implants is effective osseointegration. Surface modification of titanium-based biomedical implants is one strategy to enhance implant integration and performance, by specifically addressing the physical and chemical environment required for osseointegration without compromising the properties of the bulk implant material.

The porosity present in porous metallic titanium coatings on Ti-based implant surfaces formed with the methods herein may also be filled with other agents (e.g., growth factors) to further promote osseointegration. Antimicrobial agents may also be introduced into such porosity in the porous metallic titanium films.

Unlike certain prior processes for generating nanostructured surfaces on titanium-bearing substrates (such as biomedical implants), the methods herein are capable of providing a high concentration of nanoscale pores and protuberances of tailorable sizes on titanium-bearing surfaces. Because the present methods are not line-of-sight processes, the present methods are also capable of generating such nanoscale pores and protuberances on all exposed surfaces, including internal surfaces, of complex-shaped, titanium-bearing structures (such as 3-D printed porous Ti—Al—V alloys). Furthermore, the present methods can yield nanostructured titanium-bearing composite surfaces on titanium-bearing substrates, which can be used to tailor the chemical and mechanical behavior of such surfaces (e.g., for enhanced osseointegration of biomedical implants, or for enhanced stiffness).

While the invention has been described in terms of specific embodiments, it is apparent that other forms could be adopted by one skilled in the art. For example, the methods are applicable for processing titanium-bearing bodies have various physical configurations, and materials and processes/methods other than those noted could be used. Therefore, the scope of the invention is to be limited only by the following claims.

1. A method of forming micro- and/or nano-structures on a surface of a device, the method comprising:
exposing the surface of the device having an initial microstructure to an oxidizing environment at a first elevated temperature so as to form a first oxide scale on the surface;
exposing the first oxide scale to a reducing agent at a second elevated temperature so as to convert or partially convert the first oxide scale into a composite scale comprising a second oxide and a first metal; and
exposing the composite scale to a dissolution agent that selectively dissolves part or all of the second oxide so as to yield a porous surface layer comprising the first metal.

2. The method of claim 1, wherein the device is nonporous.

3. The method of claim 1, wherein the device is porous, the surface includes internal and external surfaces, and the internal surfaces are defined by the porosity of the device.

4. The method of claim 1, wherein the first oxide scale comprises titanium oxide.

5. The method of claim 1, wherein the reducing agent is selected from the group consisting of magnesium, calcium, strontium, barium, lithium, sodium, potassium, and rubidium.

6. The method of claim 1, wherein the first metal is titanium.

7. The method of claim 1, wherein the second oxide is selected from the group consisting of magnesium oxide, calcium oxide, strontium oxide, barium oxide, lithium oxide, sodium oxide, potassium oxide, and rubidium oxide.

8. The method of claim 1, wherein the oxidizing environment is an oxygen-bearing environment.

9. The method of claim 1, wherein the device is a biomedical implant device.

10. A device produced by a method comprising:
exposing the surface of an initial device having an initial microstructure to an oxidizing environment at a first elevated temperature so as to form an external first oxide scale;
exposing the external first oxide scale to a reducing agent at a second elevated temperature so as to convert or partially convert the first oxide scale into a composite scale comprising a second oxide and a first metal; and
exposing the composite scale to a dissolution agent that selectively dissolves part or all of the second oxide so as to yield a porous surface layer comprising the first metal.

11. The device of claim 10, wherein the device is nonporous.

12. The device of claim 10, wherein the device is porous, wherein the surface includes internal and external surfaces, wherein the internal surfaces are defined by the porosity of the device.

13. The device of claim 10, wherein the first oxide scale comprises titanium oxide.

14. The device of claim 10, wherein the reducing agent is selected from the group consisting of magnesium, calcium, strontium, barium, lithium, sodium, potassium, and rubidium.

15. The device of claim 10, wherein the first metal is titanium.

16. The device of claim 10, wherein the second oxide is selected from the group consisting of magnesium oxide, calcium oxide, strontium oxide, barium oxide, lithium oxide, sodium oxide, potassium oxide, and rubidium oxide.

17. The device of claim 10, wherein the oxidizing environment is an oxygen-bearing environment.

18. The device of claim 10, wherein the device is a biomedical implant device.

19. The device of claim 18, wherein the device comprises titanium, a titanium alloy, and/or titanium oxide.

20. The device of claim 10, wherein the average diameter of the pores in the porous surface layer is about 1 nm to about 300 nm.
